(19) United States
(12) Patent Application Publication
(10) Pub. No.: US 2008/0228009 A1
(43) Pub. Date: Sep. 18, 2008

(54) Process for Preparing 1,1,4,4-Tetraalkoxybut-2-Ene Derivatives

(75) Inventors: Ingo Richter, Schwetzingen (DE); Hermann Putter, Neustadt (DE); Ulrich Griesbach, Mannheim (DE); Till Gerlach, Ludwigshafen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 11/996,547

(22) PCT Filed: Jul. 31, 2006
(86) PCT No.: PCT/EP06/64845

§ 371 Date: Jan. 23, 2008

(30) Foreign Application Priority Data
Aug. 4, 2005 (DE) 10 2005 036 687.2

(51) Int. Cl. C07C 41/01

(52) U.S. Cl. 568/598

(57) ABSTRACT

Process for preparing 1,1,4,4-tetraalkoxybut-2-ene derivatives of the general formula (I),

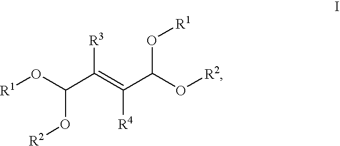

where the radicals R¹ and R² are each, independently of one another, hydrogen, C₁-C₆-alkyl, C₆-C₁₂-aryl, such as phenyl, or C₅-C₁₂-cycloalkyl or R¹ and R² together with the double bond to which they are bound form a C₆-C₁₂-aryl radical, such as phenyl, a phenyl radical substituted by one or more C₁-C₆-alkyl groups, halogen atoms or alkoxy groups or a monounsaturated or polyunsaturated C₅-C₁₂-cycloalkyl radical, R³, R⁴ are each, independently of one another, hydrogen, methyl, trifluoromethyl or nitrile, which comprises electrochemically oxidizing 1,4-dialkoxy-1,3-butadiene of the formula II

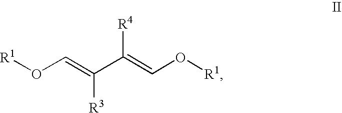

where the radicals R¹, R³ and R⁴ have the same meanings as in the formula I, in the presence of a C₁-C₆-alkyl alcohol.

The present invention relates to an electrochemical process for preparing 1,1,4,4-tetraalkoxybut-2-ene from 1,4-dialkoxy-1,3-butadiene in the presence of a C₁-C₆-alkyl alcohol by electrochemical oxidation.

Various nonelectrochemical processes for synthesizing 1,1,4,4-tetraalkoxybut-2-ene are known.

Thus, EP-A 581 097 describes the preparation of 1,1,4,4-tetramethoxybut-2-ene from 2,5-dimethoxydihydrofuran using dehydrating reagents and in the presence of acid. Electrochemical syntheses for the starting material 2,5-dihydro-2,5-dimethoxyfuran used in EP-A 581 097 are already known. Starting from furans, bromide in particular is used as advantageous oxidation catalyst (mediator) in this anodic methoxylation. Thus, DE-A-27 10 420 and DE-A-848 501 describe the anodic oxidation of furans in the presence of sodium bromide or ammonium bromide as electrolyte salts. Disadvantages of this two-stage synthesis of 1,1,4,4-tetramethoxybut-2-ene is the difficult-to-handle furan, the use of bromide as mediator, of the dehydrating agents and the formation of the by-product 1,1,2,5,5-pentamethoxybutane.

A synthesis starting from furan and bromine is disclosed in U.S. Pat. No. 3,240,818. In this process, too, furan has to be handled. Bromine is not only a very expensive oxidant, but it is difficult and costly to dispose of properly.

It was therefore an object of the invention to provide an electrochemical process for preparing tetra-1,1,4,4-alkoxybut-2-ene derivatives which is economical and gives the desired product in high yield and with good selectivity.

We have accordingly found a process for preparing 1,1,4,4-tetraalkoxybut-2-ene derivatives of the general formula (I),

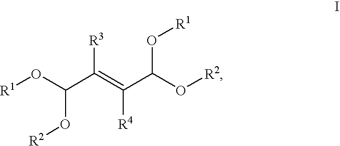

where the radicals R¹ and R² are each, independently of one another, hydrogen, C₁-C₆-alkyl, C₆-C₁₂-aryl, such as phenyl, or C₅-C₁₂-cycloalkyl or R¹ and R² together with the double bond to which they are bound form a C₆-C₁₂-aryl radical, such as phenyl, a phenyl radical substituted by one or more C₁-C₆-alkyl groups, halogen atoms or alkoxy groups or a monounsaturated or polyunsaturated C₅-C₁₂-cycloalkyl radical, R³, R⁴ are each, independently of one another, hydrogen, methyl, trifluoromethyl or nitrile, which comprises electrochemically oxidizing 1,4-dialkoxy-1,3-butadiene of the formula II

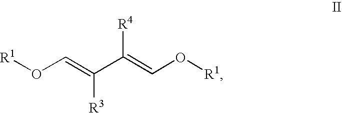

where the radicals R¹, R³ and R⁴ have the same meanings as in the formula I, in the presence of a C₁-C₆-alkyl alcohol. The radical R¹ is preferably a methyl radical.

All possible diastereomers, enantiomers and trans/cis isomers, stereoisomers and mixtures thereof of the compounds of the formulae I and II are intended to be encompassed, in particular, therefore, not only the pure diastereomers, enantiomers and isomers but also the corresponding mixtures.

1,4-Dialkoxy-1,3-butadienes are significantly cheaper than the furan used as starting material in the processes of the prior art. Owing to a higher boiling point of the 1,4-dialkoxy-1,3-butadienes, the cooling required during the reaction is also reduced and higher reaction temperatures become possible. An important further advantage of this starting material is its significantly lower toxicity. 1,4-Dimethoxy-1,3-butadienes are known per se. 1,4-Dimethoxy-1,3-butadiene can be prepared by methylation of 1,4-butynediol to 1,4-dimethoxy-2-butyne and rearrangement of this, as described, for example, in L. Brandsma in Synthesis of Acetylenes, Allenes and Cumulenes, Elesevier Ltd. 2004, p. 204, and P. E. van Rijn et al. J. R. Neth. Chem. Soc. 100, 198, 372-375. As described by H. Hiranuma et al., J. Org. Chem. 1982, 47, 5083-5088, an isomer mixture of cis,cis/cis,trans/trans,trans\tilde{-}(59±5):(35±5):(6±3)-1,4-dialkoxy-1,3-butadiene is obtained after the work-up and this is preferably used in the process of the invention. The preparation of the 1,4-dialkoxy-1,3-butadienes substituted in the 2 and 3 positions is carried out analogously.

In the electrolyte, the C₁-C₆-alkyl alcohol is used in an equimolar amount, based on the 1,4-dialkoxy-1,3-butadiene derivative of the general formula (II), or in an excess of up to 1:20 and then serves simultaneously as solvent or diluent for the resulting compound of the general formula (I). Preference is given to using a C₁-C₆-alkyl alcohol, very particularly preferably methanol.

If appropriate, customary cosolvents are added to the electrolysis solution. These are the inert solvents having a high oxidation potential which are generally customary in organic chemistry. Examples which may be mentioned are dimethylformamide, dimethyl carbonate, acetonitrile and propylene carbonate.

The electrolyte salts comprised in the electrolysis solution are generally at least one compound selected from the group consisting of potassium, sodium, lithium, iron, alkali metal, alkaline earth metal, tetra(C₁-C₆-alkyl)ammonium salts, preferably tri(C₁-C₆-alkyl)methylammonium salts. Possible counterions are sulfate, hydrogensulfate, alkylsulfates, arylsulfates, halides, phosphates, carbonates, alkylphosphates, alkylcarbonates, nitrate, alkoxides, tetrafluoroborate or perchlorate.

Furthermore, the acids derived from the abovementioned anions are possible as electrolyte salts.

Preference is given to methyltributylammonium methylsulfate (MTBS), methyltriethylammonium methylsulfate or methyltripropylmethylammonium methylsulfate.

In addition, ionic liquids are also suitable as electrolyte salts. Suitable ionic liquids are described in “Ionic Liquids in Synthesis”, edited by Peter Wasserscheid, Tom Welton, Verlag Wiley VCH publishers, 2003, Chapter 3.6, pages 103-126.

The process of the invention can be carried out in all customary types of electrolysis cells. It is preferably carried out continuously using undivided flow-through cells.

Particularly useful electrolysis cells are those in which the anode space is separated from the cathode space by a membrane or by a diaphragm. Undivided bipolar capillary cells or plate stack cells in which the electrodes are configured as plates and are arranged in a parallel fashion (cf. Ullmann's Encyclopedia of Industrial Chemistry, 1999 electronic release, Sixth Edition, VCH-Verlag Weinheim, Volume Electrochemistry, Chapter 3.5. special cell designs and Chapter 5, Organic Electrochemistry, Subchapter 5.4.3.2 Cell Design) are very particularly useful. Such electrolysis cells are also described, for example, in DE-A-19533773.

The current densities at which the process is carried out are generally from 1 to 20 mA/cm², preferably from 3 to 5 mA/cm². The temperatures are usually from −20 to 55° C., preferably from 20 to 40° C. The process is generally carried out at atmospheric pressure. Higher pressures are preferably employed when the process is to be carried out at higher temperatures in order to avoid boiling of the starting compounds or cosolvents.

Suitable anode materials are, for example, graphitic materials, noble metals such as platinum or metal oxides such as ruthenium or chromium oxide or mixed oxides of the type RuOₓTiOₓ, metals such as lead or nickel or boron-doped diamond. Preference is given to graphite and platinum. Preference is also given to anodes having diamond surfaces.

Possible cathode materials are, for example, iron, steel, stainless steel, nickel, lead, mercury or noble metals such as platinum, boron-doped diamond and also graphite or carbon materials, with graphite being preferred.

Very particular preference is given to the system graphite as anode and cathode.

After the reaction is complete, the electrolysis solution is worked up by generally known separation methods. For this purpose, the electrolysis solution is generally firstly brought to a pH of from 8 to 9, subsequently distilled and the individual compounds are obtained separately in the form of various fractions. Further purification can be carried out by, for example, crystallization, distillation or chromatography.

### EXAMPLES

### Example 1

### 1,1,4,4-tetramethoxybut-2-ene

| | |
|---|---|
| Apparatus: | Undivided plate stack cell having 6 graphite |
| | electrodes (diameter: 65 mm, spacing: 1 mm, 5 gaps) |
| Anode and | Graphite |
| cathode: | |
| Electrolyte: | 47 g of a mixture of trans,trans-, trans,cis- and |
| | cis,cis-1,4-dimethoxybutadiene |
| | 20 g of methyltributylammonium methylsulfate |
| | (MTBS) 717 g of methanol |
| | Electrolysis using 2.5 F/mol of 1,4-dimethoxy- |
| | 1,3-butadiene |
| Current density: | 3.4 A dm⁻² |
| Temperature: | 24° C. |

In the electrolysis under the conditions indicated, the electrolyte was pumped through the cell via a heat exchanger at a flow rate of 250 l/h for 5 hours.

After the electrolysis was complete, the electrolysis solution was freed of methanol by distillation and the residue was distilled at 54-64° C. and 2 mbar. This gave 46 g of 1,1,4,4-tetramethoxybut-2-ene, corresponding to a yield of 62%. The selectivity was 84%.

1. A process for preparing 1,1,4,4-tetraalkoxybut-2-ene derivatives of the general formula (I), 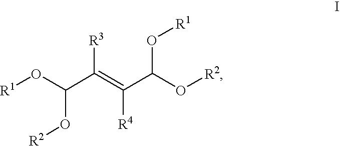
where the radicals R¹ and R² are each, independently of one another, hydrogen, C₁-C₆-alkyl, C₆-C₁₂-aryl or C₅-C₁₂-cycloalkyl or R¹ and R² together with the double bond to which they are bound form a C₆-C₁₂-aryl radical, a phenyl radical substituted by one or more C₁-C₆-alkyl groups, halogen atoms or alkoxy groups or a monounsaturated or polyunsaturated C₅-C₁₂-cycloalkyl radical, R³, R⁴ are each, independently of one another, hydrogen, methyl, trifluoromethyl or nitrile, which comprises electrochemically oxidizing 1,4-dialkoxy-1,3-butadiene of the formula II 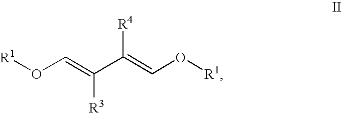
where the radicals R¹, R³ and R⁴ have the same meanings as in the formula I, in the presence of a C₁-C₆-alkyl alcohol.

2. The process according to claim 1, wherein the aliphatic C₁-C₆-alkyl alcohol is methanol.

3. The process according to claim 1, wherein at least 1 mol of alkyl alcohol is used per mole of the 1,4-dialkoxy-1,3-butadiene of the general formula (II).

4. The process according to claim 1 carried out in an electrolyte comprising sodium, potassium, lithium, iron, tetra(C₁-C₆-alkyl)ammonium salts with sulfate, hydrogensulfate, alkylsulfates, arylsulfates, halides, phosphates, carbonates, alkylphosphates, alkylcarbonates, nitrate, alkoxides, tetrafluoroborate, hexafluorophosphate or perchlorate as counterion or ionic liquids as electrolyte salt.

5. The process according to claim 1 carried out in a bipolar capillary cell or plate stack cell or in a divided electrolysis cell.
